(19) United States
(12) Patent Application Publication
(10) Pub. No.: US 2026/0207968 A1
(43) Pub. Date: Jul. 23, 2026

(54) Method and Device for Treating Damaged Tissue

(71) Applicants: EndoCellutions, Inc., Marshfield, MA (US); IPG Photonics Corporation, Oxford, MA (US)

(72) Inventors: Andrew McGillicuddy, Humarock, MA (US); Ilya Yaroslavsky, North Andover, MA (US); Emil Sobol, Framingham, MA (US); Andrey Baskov, Moscow (RU)

(21) Appl. No.: 19/380,135

(22) Filed: Nov. 5, 2025

Related U.S. Application Data
(63) Continuation of application No. 17/414,740, filed on Dec. 18, 2019, now abandoned.
(60) Provisional application No. 62/781,439, filed on Dec. 18, 2018.

(51) Int. Cl. A61N 5/067; A61B 17/00; A61N 1/32; A61N 1/36; A61N 5/06

(52) U.S. Cl. CPC A61N 5/067; A61N 5/0601; A61N 5/0613; A61B 2017/00172; A61N 1/326; A61N 1/327; A61N 1/36021; A61N 1/36031; A61N 1/36034; A61N 1/36042; A61N 2005/063

(57) ABSTRACT

A device and associated method for treatment of tissue in a joint of a mammal includes an energy source and an energy-delivery implement coupled to the energy source. The energy-delivery implement has a distal portion configured to be inserted into the joint, the distal portion having at least one energy-emitting portion. A control module causes the energy source to produce energy in a pre-defined treatment sequence, the sequence comprising at least two pulses of energy separated by an interval. The energy-delivery implement is configured to emit the energy at the distal portion, to create a zone of thermal stress within target tissue, without causing substantial coagulation of the target tissue, wherein the size of the thermal stress zone is substantially less than the size of the target tissue being treated. A biologic can be injected or otherwise delivered into the joint before, during, or after delivery of the energy.

### RELATED APPLICATIONS

This application a continuation of U.S. application Ser. No. 17/414,740, filed on Jun. 16, 2021, which is the U.S. National Stage of International Application No. PCT/US2019/067255, filed Dec. 18, 2019, which designates the U.S., published in English, and claims the benefit of U.S. Provisional Application No. 62/781,439, filed Dec. 18, 2018. The entire teachings of the above applications are incorporated herein by reference.

### BACKGROUND

The use of various biologics to treat chronic pain associated with arthritis and other disease has been an area of clinical study. Commonly used biologics are autologous platelet rich plasma (PRP), marrow, fat and some derivatives of amniotic tissue. More invasive procedures that alter the matrix of joint tissue, such as the practice of micro-fracture in the knee, have been associated with formation of type 1 collagen and fibrotic cartilage, but not hyaline cartilage. Micro-fracture is a procedure where holes are drilled through the cartilage and cortical bone into the knee. The marrow that infiltrates the joint from this procedure is thought to contribute to fibrotic cartilage tissue formation. While fibrotic cartilage provides some temporary relief, it wears out quickly, unlike hyaline cartilage that has type 2 collagen as its substrate. Simply injecting marrow (or other biologics) without drilling through the cortical bone or using some other technique to alter the matrix of the tissue will typically not result in any new tissue formation (fibrotic or hyaline) but has been associated with pain relief. In short, injected biologics at best reduce pain and may delay total joint replacement but they do not repair hyaline like cartilage or ligaments in a joint. Thus, simply injecting biologics into joints provide some success in the resolution of pain and more invasive matrix altering techniques cause fibrotic cartilage formation, but these procedures have not been reported as being successful in disease modification, such as the repair of hyaline like cartilage or regeneration of ligaments.

Marrow has been used as an adjunct to treat acute cartilage defects as part of a surgical intervention. Cartilage grafts made from cadavers or animal tissue are hydrated in marrow. Surgically, necrotic tissue is debrided and the marrow hydrated graft is sown into place on the surface of the joint. Alternatively, culture expanded chondrocytes are seeded onto the graft (often referred to as a MACI procedure). Adding marrow or chondrocytes to theses acute surgical procedures has been reported as being beneficial. The degree of hyaline like cartilage formed over the graft is controversial with many in the medical community believing that the graft results primarily in fibrotic cartilage formation. The long-term success of these procedures is then questionable. Acute injuries, created surgically or otherwise, are biologically very different from chronic disease. Acute injuries are often created in animal models as a first line test. A healthy animal that artificially has a defect created is different biologically from a person who has a chronic condition, i.e., a condition that developed over many years. Chronic conditions have other confounding factors, such as co-morbidities (i.e., diabetes) or other adjacent tissue that is also diseased. These factors and others make it difficult to draw conclusions from animal work as it will relate to humans. However, it is generally thought that thicker fibrotic cartilage is better than thinner fibrotic cartilage. While fibrotic cartilage wears away more rapidly than hyaline cartilage, the thicker it is, the longer it is likely to last.

### SUMMARY

Described is a therapy, including associated devices and methods, to regenerate damaged tissue, which combines delivering energy and delivery a biologic to the damaged tissue.

An autologous biologic can be harvested from an area different from the damaged tissue in need of regeneration. The autologous biologic can be harvested by using a syringe and cannula, the cannula being introduced into the body to access the biologic. The autologous biologic can be harvested and transplanted during the same procedure and before or after application of energy delivered to the tissue. The autologous biologic can be transplanted to an area in need of regeneration and energy can be delivered over a fiber, the fiber being introduced into the body and positioned near the area in need of regeneration. Energy can be delivered to two separate locations to treat both the tissue damage caused by harvesting the biologic and the area in need of regeneration where the biologic is transplanted. The fiber can be introduced to the body via a cannula or can be used during an open procedure. The cannula that is used to harvest and/or deliver the biologic can be the same cannula used to introduce the fiber. The placement of the fiber can be assisted by imaging technology such as ultra-sound or an arthroscope. The tip of the fiber can be capped with a micro-array to allow multiple spots to be treated simultaneously or otherwise capped to allow the direction of the energy to be at an angle (e.g., 90 degrees) to the orientation of the fiber, i.e., a side firing fiber.

A device for treatment of tissue in a joint of a mammal includes an energy source and an energy-delivery implement coupled to the energy source. The energy-delivery implement has a distal portion configured to be inserted into the joint, the distal portion having at least one energy-emitting portion. The device includes a control module that causes the energy source to produce energy in a pre-defined treatment sequence, the sequence comprising at least two pulses of energy separated by an interval. The energy-delivery implement is configured to emit the energy at the distal portion, to create a zone of thermal stress within target tissue, without causing substantial coagulation of the target tissue, wherein the size of the thermal stress zone is substantially less than the size of the target tissue being treated.

The energy can be optical energy and the energy-delivery implement can include an optical fiber. The energy-delivery implement can be configured to create plural thermal stress zones simultaneously. For example, the energy-delivery implement can include a bundle of optical fibers or a multi-core optical fiber. In another example, a micro-array is attached to an end of the fiber allowing for plural thermal stress zones to be created simultaneously.

The energy source can be a directed-energy source and can be, for example, a laser.

A method to treat damaged tissue in a joint of a mammal include providing a treatment device including an energy source and an energy-delivery implement. The energy-delivery implement is inserted into the joint. An energy-emitting portion of the implement is positioned in the proximity of a first target spot in a target zone of the damaged tissue. The energy source of treatment device is activated to deliver a treatment sequence of energy at the first target spot, the sequence comprising at least two pulses of energy separated by an interval, to create a zone of thermal stress within target tissue, without causing substantial coagulation of the tissue. Next, the energy-delivery implement is relocated to position the energy-emitting portion in the proximity of another target spot of the target zone, located from the first target spot at a distance not shorter than a radius of the zone of thermal stress. The steps of activating the energy source and relocating the energy-delivery implement are repeated until energy is delivered to all target spots of the target zone.

The distance between two neighboring target spots can exceed 1 mm. At least two target spots can be treated simultaneously.

To treat multiple target spots simultaneously, the energy-delivery implement can include, for example, a bundle of fibers or a micro-array.

The duration of time the energy is delivered at a single target spot can be in a range of about 10 seconds to about 5 minutes. For example, the duration can be about 50 seconds.

The energy delivered can be turned on and off while the energy-delivery implement is held at a single location with a time interval between when the energy is turned off and when it is turned on being in a range of about 1 second to about 1 minute. For example, the time interval can be about 10 seconds.

The energy-delivery implement can include a micro-array, in which case the energy emitted can be under 3 watts of power per target spot treated by the micro-array and the size can be 900 microns or less for each target spot.

The pulse repetition frequency can be in a range of about 0.2 Hz to about 3.2 Hz

The energy source can be a laser, in which case the wavelength of the energy can be in a range of about 0.6 microns to about 2.1 microns. For example, the wavelength can be in a range of 1.3 microns to 1.65 microns.

The energy-delivery implement can include a micro-array head that contains plural lenses with centers positioned greater than 0.05 millimeters apart and less than 5 millimeters apart. For example, the lens centers are about 0.5 millimeters apart.

The energy-delivery implement can include an array of plural diffractive elements, centers of the elements positioned greater than 0.05 millimeters apart and less than 5 millimeters apart. For example, the centers of the diffractive elements can be about 0.5 millimeters apart.

The method can further include identifying a location of the damaged tissue in the joint using a diagnostic device and identifying a target zone to be treated at the location of the damaged tissue, the target zone containing plural target spots. For example, identification of the target zone can be accomplished by a human operator.

The method can further include injecting a biologic into the joint. Before injecting a biologic in the joint, a location of the damaged tissue in the joint can be identified using a diagnostic device. Additionally, a target zone to be treated at the location of the damaged tissue can be identified before injecting the biologic, the target zone containing plural target spots.

A biologic can be injected into the joint within six months of delivering the energy to the joint. The biologic can include autologous tissue. Energy can be used to treat the area where the autologous tissue is sourced from and where the autologous tissue is transplanted into. The autologous tissue can be marrow aspirate, adipose aspirate, or platelet rich plasma. For example, the biologic can include autologous tissue sourced from bone marrow, and the biologic can be delivered on a medullary side of the joint and an articular side of the joint.

The energy-delivery implement can include a fiber, a distal end of the fiber including the energy-emitting portion. Optical feedback or temperature feedback can be used to monitor tissue conditions near the distal end of the fiber.

A method to treat a tissue graft being placed in a joint of a mammal during a surgical procedure includes impregnating an exogenous tissue graft with cells prior to the graft being placed in the joint, placing a fiber in proximity of the graft; and delivering energy in a timed, pulsed, and automated sequence over the fiber to the graft, each sequence delivering energy below than or close to that required to coagulate tissue.

A tip of the fiber can be placed within 10 mm of the graft when energy is being delivered. The energy can be delivered prior to the graft being placed in the joint

The cells that are used to impregnate the graft can be autologous cells and can be sourced from the mammal during the surgical procedure. For example, the mammal can be a human patient and the cells are sourced from the human patient. Alternatively, the mammal can be a human patient and the cells are sourced from another mammal.

A method to treat damaged tissue in a joint of a mammal includes inserting the energy-delivery implement of a treatment device into the joint, positioning an energy-emitting portion of the energy-delivery implement in the proximity of a first target spot in a target zone of the damaged tissue, activating the treatment device to deliver a treatment sequence of energy at the first target spot, the sequence comprising at least two pulses of energy separated by an interval, to create a zone of thermal stress within target tissue, without causing substantial coagulation of the tissue, and delivering a biologic into the joint.

The method can further include relocating the energy-delivery implement to position the energy-emitting portion in the proximity of another target spot of the target zone, located from the first target spot at a distance not shorter than a radius of the zone of thermal stress. The activating and relocating can be repeated until energy is delivered to all target spots of the target zone.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a block diagram of a directed-energy treatment device according to an example embodiment.

FIG. 1B illustrates a single fiber that can be used with the device of FIG. 1A to transmit energy into a joint space.

FIG. 1C shows various hand pieces that can be used to deliver topical energy that can supplement the energy delivered interstitially.

FIG. 2 is a graph illustrating an example pulse sequence that can be used for treatment of tissue. Here Tₚ is the pulse period (pulse duration+interval between pulses) of pulses within a group, and T_{g} is the group period (total duration of group+interval between groups). The group period is substantially longer than the pulse period. Preferably, at least two groups of pulses are used for treatment. Most preferably, three groups of pulses are used for treatment.

FIG. 3 illustrates an energy delivery device including an optical fiber and a micro array according to an example embodiment.

FIG. 4 illustrates an energy delivery device including a bundle of optical fibers according to an example embodiment.

FIG. 5 illustrates an aspiration device including a cannula to source a biologic such as bone marrow for use with embodiments of the invention.

FIG. 6 illustrates a side firing fiber that can be used as an energy-delivery implement according to an example embodiment.

FIG. 7 is a schematic illustration of a joint including bone marrow edema and insertion of treatment devices.

FIGS. 8A and 8B illustrate a cartilage graft procedure.

FIGS. 9A and 9B show example of temperature distribution and temperature dynamics, respectively, for cartilage being illuminated by a laser in vitro.

FIGS. 10A-10D illustrate surgically created defects in cartilage of the medial femoral condyle in an animal on the treatment side (FIGS. 10A, 10C) and the control side (FIGS. 10B, 10D).

FIG. 11 is a microscopic image of cells in the treatment area.

### DETAILED DESCRIPTION

A description of example embodiments follows.

Use of topical lasers (cold laser therapy) has been in practice for several years. The mechanism of action of these lasers is to stimulate repair without damaging tissue. The heat generated is therefore below what is needed to coagulate tissue. The results of this therapy have been mixed. Part of the explanation for these mixed results is that the energy from the laser device does not penetrate deep into the tissue. Rather, the light is scattered with most of the energy absorbed by tissue covering the injury. For example, joint tissue can be very difficult to treat with a topical laser due to the depth of the injury. This depth can be varying depending on the size of the patient and the joint impacted. The amount of energy needed to have a clinical effect will be different depending on the size of the damaged area. Topical delivery of energy to interstitial tissue is difficult because no precise way is known how to customize the power and time to deliver an effective dose to the damaged tissue because to many variables are present. Lasers that are applied over a fiber and interstitially using a cold laser energy level have been studied. These lasers employ various means of delivery either through an open procedure or a cannula. None of these devices are commercially available to patients today because the experiments in the literature have not been reduced to practice so that a clinician can use a cold laser interstitially to treat patients. A method to improve on this therapy is to deliver the energy to the damaged tissue over a fiber in an automated fashion.

Medical lasers have been used where energy is delivered over a fiber to coagulate or cut tissue. These lasers are often continuous wave or pulsed intermittingly to allow the generation of the required heat to ablate tissue. Use of these medical lasers is often combined with guidance/visualization tools so that the surgeon can visualized the tissue being ablated. Papers have been published on how to deliver energy over a fiber using various frequencies and energy levels, pulsed and timed to optimize the potential regenerative capability of using a fiber to deliver energy to tissue in need of repair. Major obstacles revolve around how to know the location of the tip of the fiber to ensure accurate dispersion of the energy. A manner to precisely treat the damaged tissue is required that includes automation and efficiency to treat various joints and various sized lesions. Precise delivery of energy to a joint is considered beneficial to success. Knowing at what the distance the energy source is located from the damaged tissue is useful to customizing the amount of energy delivered. Knowing the distance between different spots where the energy is delivered is also useful. Because these shortcomings have not been addressed, none of the work using lasers to regenerate tissue has led to widespread use of lasers to regenerate tissue where the energy is delivered over a fiber and to interstitial tissue.

The most common pathological feature of post-traumatic osteo-arthritis (OA) is a lesion of the articular cartilage plate. If such lesions are relatively large (more than 3 mm in size) and superficial (partial-thickness defects that do not reach the bone), they do not repair without external intervention. More deep, full-thickness injuries are usually covered with fibrous tissue or trabecular bone. Previous studies have reported mixed results on using biologics or lasers to treat these defects.

As the work of Sobol and colleagues has demonstrated in animal models of acute OA, the creation of micro-channels into the surgically created damaged tissue using directed energy delivered over a fiber will lead to hyaline cartilage formation as evidenced by type 2 collagen. (See., e.g., Emil Sobol, Olga Baum, Anatoly Shekhter, Sebastian Wachsmann-Hogiu, Alexander Shnirelman, Yulia Alexandrovskaya, Ivan Sadovskyy, Valerii Vinokur, “Laser-induced micropore formation and modification of cartilage structure in osteoarthritis healing,” J. Biomed. Opt. 22(9), 091515 (2017), doi: 10.1117/1.JBO.22.9.091515.) What is unique in the Sobol et. al work is that no bulk acute injury is created because the power delivered is not sufficient to coagulate large volume of tissue. This low power is thought to prevent scar formation by preventing the body's natural reparative processes from beginning. This work looks to improve on micro fracture (micro fracture drills holes into subchondral bone) by creating nanometer sized channels that do not penetrate bone and does not coagulate tissue. The energy delivered in the Sobol et al. work created channels, an effect similar to micro-fracture, but on a smaller scale.

Prior approaches teach away from using a biologic because they consider marrow creates fibrotic scar tissue when combined with energy that causes micro-channels into cartilage. Marrow infiltrating the joint space is thought to enhance fibrotic tissue formation in the micro-fracture protocol. One of the body's natural reparative processes is mobilizing marrow to the site of the injury. Sobol et al. specifically teach away from adding a biologic for this reason, to avoid fibrotic cartilage formation. Sobol et al. speculated in their work that adding a biologic such as marrow to the procedure of delivering energy to cartilage would work at cross purposes to the formation of hyaline cartilage. Their reasoning was that the body's natural response to injury is bleeding which brings in cells responsible for rapid tissue formation similar to scar tissue that forms when skin is damaged. This rapid tissue formation, for example, results from micro-fracture, where bleeding from the sub chondral distal end of the bone marrow infiltrates the joint space through the holes drilled. In fact, great care was taken by Sobol et al. not to deliver energy sufficient to stimulate cells below the cartilage layer because of this fibrotic tissue formation concern. Sobol and colleagues teach away from using biologics to supplement this procedure. The defects created in the animals by Sobol et al. were in the load bearing section of the knee, i.e. the medial femoral condyle. It is recognized that load bearing cartilage has a different healing potential (harder to heal) than non-load bearing. Also, acute injuries (surgically created or otherwise) are different than chronic disease that forms over time. Lastly, disease that results from unusual wear due to removal of tissue such as meniscal or ligament or frayed cartilage does not regenerate. Often, anterior cruciate ligament (ACL) and medial collateral ligament (MCL) tears or weakening cause pain and unusual wear of cartilage. Finally, bone marrow edema often accompanies joint disease and is a further source of a patient's chronic pain. Stated simply, joint disease in human beings is more complicated than treating a defined surgical defect in an animal. There are several causes of pain and tissues that are degraded in diseased joints. The work by Sobol et al. did not demonstrate disease modification with their protocol in chronic OA of a joint in a human, as evidenced by the standard measure of magnetic resonance imaging (MRI).

The use of biologics in joints, when not combined with drilling or other energy delivery that is meant to alter the cartilage matrix, will alleviate pain and inflammation; however, because there is no altering of the cartilage matrix during these procedures, no formation of fibrotic cartilage is observed. In addition, prior approaches have used a single fiber that treats a single narrow spot. This works in experiments, where the defect is created ahead of time but in clinical practice, this makes the procedure tedious and makes it difficult for the clinician to keep track of different spots treated.

Prior studies have demonstrated that supplementing acute injuries in the greatest load bearing section of a joint using marrow assists in tissue formation, that tissue formation being dominated by fibrotic cartilage. Using marrow in a non-acute setting (injection therapy) does not result in tissue formation but has been reported to result in pain reduction. Using low power pulsed energy delivered in close proximity to damaged cartilage, a power density below what is needed to coagulate tissue, shows the development of hyaline cartilage in animal models where the defect was surgically created. This protocol success is thought to be the result of matrix altering effects without any tissue damage. Tissue damage, it is believed, stimulates a healing cascade, mobilization of marrow to the joint and the formation of fibrotic cartilage.

It was discovered through experimentation that adding marrow to the procedure where low level energy below what coagulates tissue is delivered to damaged joint tissue, either in an open procedure or arthroscopically, can lead to improved hyaline cartilage formation without fibrotic cartilage formation. This result is opposite of what was expected because it was thought that cells from marrow would create fibrotic tissue. Marrow cells have demonstrated an ability to convert to chondrocytes in culture but marrow cells do not differentiate in vivo into chondrocytes. This unexpected result is thought to be the result of the altered matrix of the cartilage causing a stimulatory effect to the marrow that results in a conversion of marrow stem cells into chondrocytes that accelerate the formation of type 2 collagen of hyaline cartilage.

It was discovered through experimentation that adding marrow to the procedure where low level energy below what coagulates tissue is delivered to damaged joint tissue, either in an open procedure or arthroscopically, can lead to improved ligament formation without fibrotic tissue formation. This result was unexpected as ligaments had not been a tissue where low level energy delivered over a fiber had been reported as providing a benefit.

In reducing the procedure into practice, the procedure of delivering low level energy and biologics into a joint, improvements were required to make the procedure something that could be conducted on humans in a clinical setting. Part of the reduction to practice was using the marrow aspiration cannula as a means to provide low level energy to the marrow space which results in enhanced mobilization of stem cells to the vasculature. Higher levels of circulating stem cells have been associated with accelerated healing.

Marrow hydrated cartilage grafting or MACI procedures can be improved through addition of energy below than or close to a threshold needed to coagulate tissue.

These discoveries and techniques will become clearer in view of the illustrations and description of experiments performed.

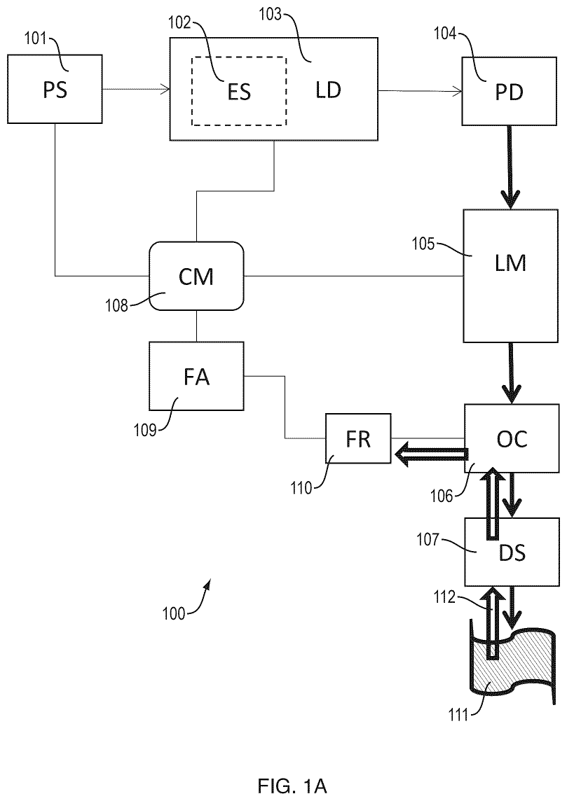

FIG. 1A is a block diagram of a directed-energy treatment device 100 according to an example embodiment. The device 100 includes a power supply (PS) 101 coupled to a laser driver (LD) 103. The laser driver 103 includes energy storage (ES) 102, e.g., capacitors, and is configured to drive pump diodes (PD) 104 which, in turn, are coupled to a laser module (LM) 105, e.g., a fiber laser. Optical coupler (OC) 106 couples the laser module 105 to a delivery system (DS) 107. The delivery system 107 includes an energy delivery implement to delivery energy to the target tissue 111. As further described herein, the energy delivery implement can be a fiber or a bundle of fibers and may include a micro array. An optical feedback signal 112 from the target tissue is transmitted via the delivery device 107 and optical coupler 106 to a feedback receiver (FR) 110. The feedback receiver 110 is coupled to the feedback analyzer (FA) 109, which is coupled a control module (CM) 108. The control module 108 is also coupled the other components of the device 100, including, as illustrated, power source 101, laser driver 103, and laser module 105. The control module 108 can be configured to control all elements of the device 100.

The device 100 illustrated in FIG. 1A provides a device for treatment of tissue in a joint of a mammal. The device includes an energy source, e.g., laser driver 103, pump diodes 104 and laser module 105, and an energy-delivery implement, e.g., delivery device 107, which is coupled to the energy source. The energy-delivery implement has a distal portion configured to be inserted into the joint, the distal portion having at least one energy-emitting portion. The control module 108 causes the energy source to produce energy in a pre-defined treatment sequence, the sequence comprising at least two pulses of energy separated by an interval. The energy-delivery implement 107 is configured to emit the energy at the distal portion, to create a zone of thermal stress within the target tissue 111 of the joint, without causing substantial coagulation of the target tissue, wherein the size of the thermal stress zone is substantially less than the size of the target tissue being treated. As further described herein, a biologic can be injected or otherwise delivered into the joint before, during, or after delivery of the energy.

The energy-delivery implement 107 can include a fiber. A distal end of the fiber can include the energy-emitting portion. Optical feedback 122 or temperature feedback can be used by the feedback analyzer 109 and/or control module 108 to monitor tissue conditions near the distal end of the fiber.

The treatment device 100 of FIG. 1A can be used in method to treat damaged tissue in a joint of a mammal. The energy-delivery implement 107 of the device 100 is inserted into the joint. An energy-emitting portion of the energy-delivery implement 107 is positioned in the proximity of a first target spot in a target zone of the damaged tissue. The energy source of treatment device, e.g., laser module 105, is activated to deliver a treatment sequence of energy at the first target spot, the sequence comprising at least two pulses of energy separated by an interval, to create a zone of thermal stress within target tissue, without causing substantial coagulation of the tissue. Next, the energy-delivery implement 107 is relocated to position the energy-emitting portion in the proximity of another target spot of the target zone, located from the first target spot at a distance not shorter than a radius of the zone of thermal stress. Activating the energy source and relocating the energy-delivery implement are repeated until energy is delivered to all target spots of the target zone.

FIG. 1B shows a single fiber 120 that can be used to transmit energy, i.e., from the laser, over the fiber and into a joint space. A distal end 122 of the fiber includes an energy-emitting portion.

FIG. 1C shows various hand pieces 130, 132, 134, 136 that can be used to deliver topical energy that can supplement the energy delivered interstitially. Choice of a particular handpiece is dictated by dimensions and individual anatomy of the treated joint.

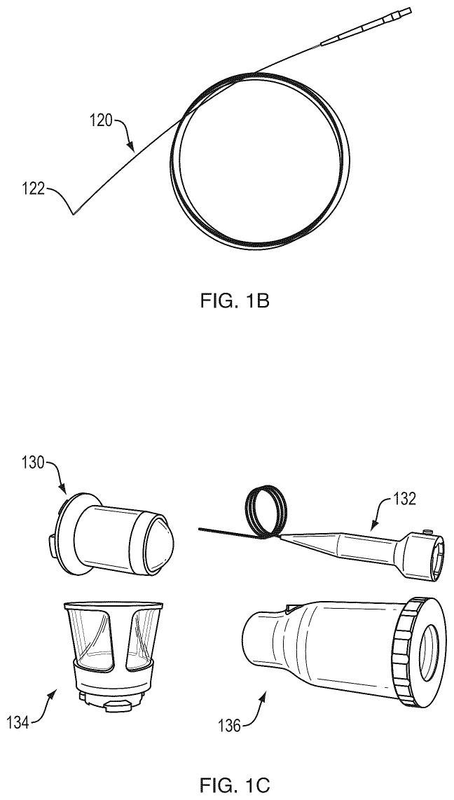

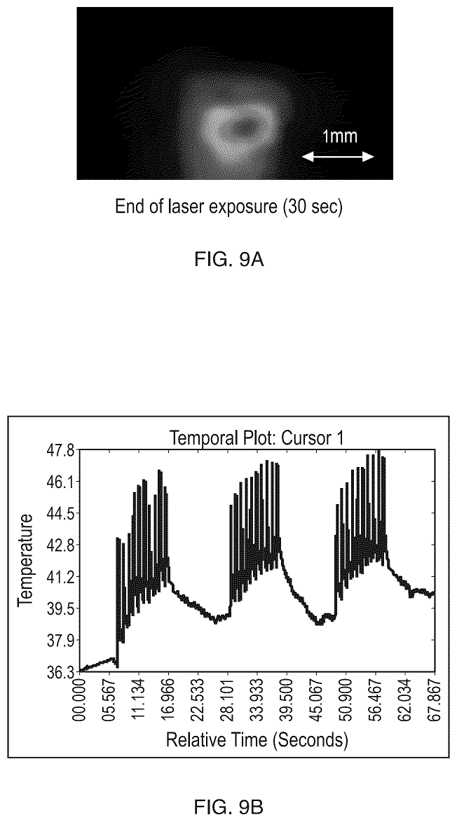

FIG. 2 is a graph illustrating an example pulse sequence 200 that can be used for treatment in embodiments of the invention. Pulse amplitude is plotted as a function of time. A single pulse 101 has amplitude Pp. Pulses 101 are arranged in groups 202. Here, Tpis the pulse period (pulse duration+interval between pulses) of pulses within a group, and T_{g} is the group period (total duration of group+interval between groups). The group period is substantially longer than the pulse period. Preferably, at least two groups of pulses are used for treatment. Most preferably, three groups of pulses are used for treatment. In the exemplary pulse structure shown, each group 202 includes three single pulses 201. The number of pulses in each group and the number of groups in a sequence may be adjusted according to the application. Another example of a pulse sequence is illustrated in FIG. 9B.

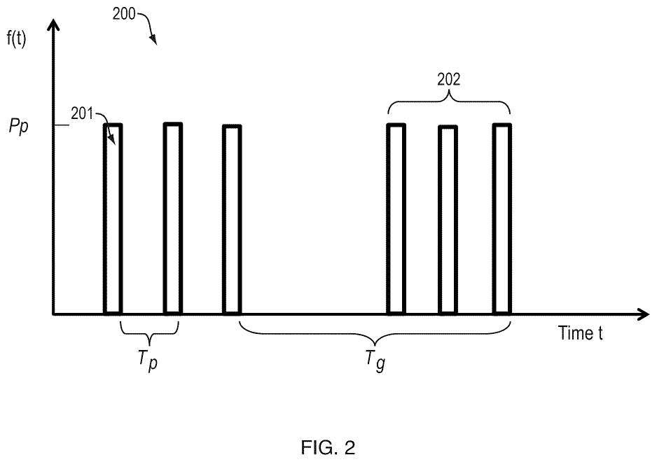

FIG. 3 depicts an exemplary embodiment of delivery system 300 with a microlens array. Energy from optical fiber 301 is distributed by microlens array 302 to illuminate multiple spots of target tissue 303. The plural lenses of the microlens array can have lens centers positioned greater than 0.05 millimeters apart and less than 5 millimeters apart. For example, the lens centers can be about 0.5 millimeters apart. Although microlenses are illustrated in FIG. 3, it will be understood that other diffractive elements can be used and arranged in a micro array.

In general, the tip of the fiber can have a micro-array that allows for more than one spot at a time to be treated. The array can use mirrors, lenses, diffractive elements, or other known methods to divide the energy created by the laser to multiple specific spots. The spots treated by the micro-array can be more than 0.25 mm apart but less than 10 mm apart. They can be 2 mm apart. Preferably, they are 0.5 mm apart. The energy delivered through the micro-array can cycle on and off and be pulsed. The micro-array can be configured to selectively delivery energy to alternating spots while not delivering energy to other spots.

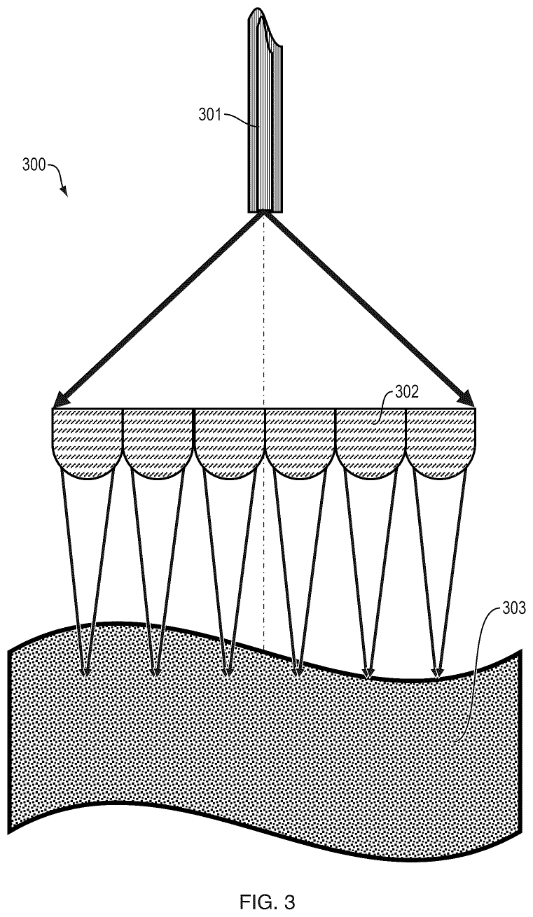

FIG. 4 depicts an exemplary embodiment of delivery system 400 with a bundle of fibers. Individual fibers 401 (1, 2, . . . , n) are bundled in a common jacket 402, each fiber configured to deliver energy to target tissue 403.

In general, multiple fibers can be bundled together and spaced so the tips of the fibers are between 0.25 mm and 10 mm apart. They can be 2 mm apart. Preferably, they are 0.5 mm apart. The bundled fibers can fire (emit radiation) simultaneously or they can fire in an alternating sequence. The sequence of when the fiber fires and is at rest and the pulsing of the laser when firing can be automated.

The energy delivered by a single fiber, bundled fiber, or micro-array does not coagulate tissue and, therefore, the temperature of the treated tissue does not increase above 70° C. Generally, the tip of the fiber (or source of the energy) is just touching or less than a millimeter from the surface of the damaged tissue as determined through an arthroscope, ultrasound, or another visualization device known in the art. Alternatively, the tip may be at a distance from the tissue (e.g., 2 mm), controlled by a spacer, and an optical component (e.g., refractive lens or diffractive element) can focus the optical energy at or below the tissue surface. The timing, pulsing, and frequency of the energy (from a laser or otherwise, e.g. ultrasound energy) is automated and the exact location of the tip of the fiber is determined by ultrasound or a scope allowing for automated settings and precise delivery of energy. Consequently, upon visual inspection, treated tissue and untreated tissue look similar immediately after application of the energy because the level of energy does not create coagulation sufficient to be visually detectable.

For example, energy is delivered to tissue from the fiber when in close proximity to the tissue. The tip of the fiber is held in place as the energy is delivered. The fiber can be held in the same place for at least 10 seconds and may be held in place up to 5 minutes. Preferably, the tip of the fiber is held in place for 40-60 seconds, more preferably for 50 seconds. The energy delivered can be pulsed during the time that the fiber is held in place. Also, the energy from the fiber can be delivered in a manner where the energy is delivered for a period of time, the energy is turned off, and then the energy is turned back on, all of this occurring while the tip of the fiber is held in a single location.

For example, the fiber can be held in one place for 50 seconds. During that time frame, energy can be delivered in a pulsed manner and then turned off and then delivered in a pulsed manner. The timeframe between when the laser (or other energy source) is shut down and when it fires in a pulsed fashion can be as little as 1 second and as long as 1 minute but is preferably 10 seconds.

For example, energy from a laser can be delivered through an optical fiber using a cannula that is also used to source the autologous biologic. The wavelength used to treat the area where the autologous biologic tissue is sourced from can be lower than 630 nm, red or near infrared for example 630-980 nm or a different spectrum, such as a range between 1.0 to 2.1 microns. The wavelength used to treat the area of damage can be different or the same as the wavelength used to treat the area the biologic is transplanted. Various times, wavelengths and pulse durations are contemplated. The area where the biologic is transplanted, for example, the joint space where damaged cartilage exists, can be treated with a combination of a wavelengths between 1.3 microns and 2.1 microns and a red or near infrared wavelength of between 630-980 nm.

The fiber can access the area in need of regeneration through a cannulated needle. Treating damaged tissue using energy from a laser with the fiber delivered over a cannula to both the area the biologic is sourced from (using a cannula for delivery of the fiber) as well as the area the biologic is transplanted (using a cannula for delivery of the fiber) will accelerate healing at both locations and improve the body's response to the therapy. The cannula used to source the biologic and deliver both the biologic and the fiber can be the same or a different cannula.

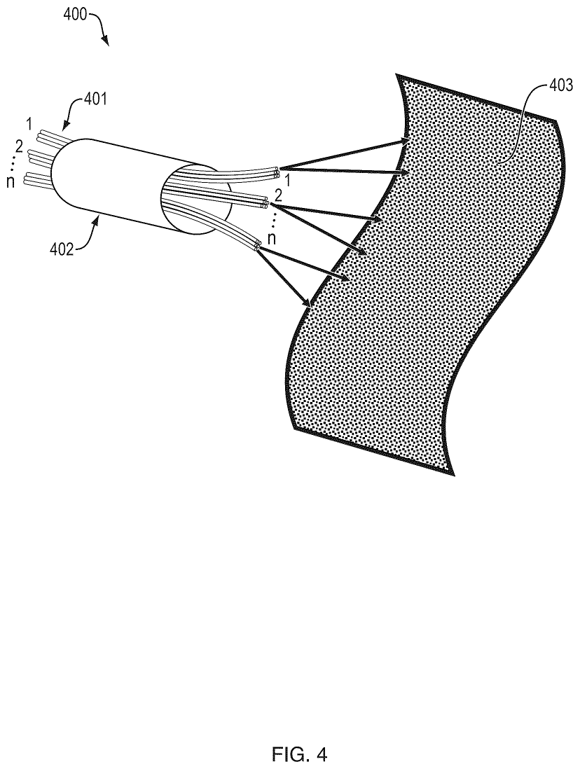

FIG. 5 depicts a bone marrow aspiration needle 500. As illustrated, the needle 500 includes an inner aspiration cannula 502, which includes one or more side holes near its distal end for aspirating of bone marrow. The aspiration cannula fits inside an introducer cannula 505, which is configured to penetrate cortical bone to access the marrow space. A screw mechanism 506 is provided to facilitate positioning of the cannulas 502, 504 within bone and retrieval of the cannulas from the bone. For aspiration, a syringe or other vacuum source can be coupled to the aspiration cannula 502 via connector 508.

Marrow can be used to supplement the creation of micro-channels that the laser and fiber create. Also, the channel created by the aspiration needle in the marrow can be used to insert the fiber into the marrow to help repair the marrow and to stimulate stem cells to exit the marrow space and home to the joint being treated.

Certain fluids and tissues absorb energy more efficiently than others. Certain fluids preferentially adhere to roughened or damaged tissue more readily than other tissue. For example, certain contrast dyes or blood adhere to damaged tissue. Coating tissue with such a material prior to the delivery of the energy can result in a more efficient therapy. Such energy can be delivered interstitial or topically.

The therapy to regenerate damaged tissue relies on pulsed energy that can be of different wavelengths produced from a laser or lasers (alternatively, LED lights or thermal light sources can be used). The energy can be delivered over a fiber or fibers. The fiber or fibers can be inserted into a targeted area where tissue damage exists in the body. The fiber or fibers can be delivered through a cannula. Different wavelengths can be generated and delivered over the fiber. A biologic can be delivered to the targeted damaged tissue prior to or after delivery of the energy by the fiber. The biologic delivered to the damaged tissue can be autologous. Energy from the laser can be used to treat the damage created by the sourcing/harvesting of the autologous biologic. Energy from the laser can be used to treat tissue damage where the biologic is transplanted. The fiber can be delivered over the same cannula used to harvest the biologic. Preferably, the harvesting of the autologous biologic and application of the energy from the laser are performed in the same day, e.g., during the same appointment or procedure. The autologous biologic can be platelet rich plasma, bone marrow aspirate, adipose tissue or any combination thereof. Allogeneic biologics such as those sourced from after birth tissue are contemplated.

One set of laser settings can include pulse repetition frequencies that can be in a range from 0.2 Hz to 1.2 Hz with a wavelength between 1.0 microns and 2.1 microns and a beam diameter between 300 and 900 microns. The peak laser power for a single fiber configuration may vary between 0.5 W and 5.0 W, preferably between 0.8 W and 1.2 W. When a bundle of fibers or a micro array is used, the peak power scales with the number of simultaneously treated spots. The temperature of the treated zone during the laser treatment (as detected by thin thermocouple and IR radiometer), can reach between 30° C. and 70° C. but preferably ca. 50° C. Interaction of laser irradiation with tissue can provide temperature gradients of an order of 50 degrees/cm to 150 degrees/cm with a preferable gradient of 100 degrees/cm at a frequency of 1.0 Hz. Preferably, one of the frequencies used can be generated by an Erbium-doped fiber laser or a Thulium-doped fiber laser or direct diode laser. Preferably, the laser radiation is delivered in bursts of 2 to 100 pulses, with interval between the bursts of pulses ranging between 3 seconds to 60 seconds, and the total number of bursts ranging between 2 and 20. Preferably, multiple locations within the target area are treated, with distance between the neighboring locations ranging from 1 mm to 20 mm.

A second, different set of laser energy can be delivered over the same or a different fiber. In one embodiment, the second set of laser energy can be delivered non-invasively through transcutaneous illumination. The radiation wavelength from this different laser setting can be between 630-980 nm, with a variable pulse repetition frequency. Preferably, one of the frequencies used can be generated by a Gallium Aluminum Arsenide laser with a wavelength of between 670 to 980 nm with dual frequency wavelengths contemplated, for example, an 810/980 nm dual frequency. A key feature of the fiber used interstitially is that the spot size is small, approximately 300 microns. Also, energy delivery optics can be added to the tip of the fiber to improve the efficiency of the delivery of the laser energy. For example, a micro array can be added to irradiate multiple spots and depths simultaneously.

The laser energy measured by wavelength, duration, and frequency, which is used to treat the tissue damage created by harvesting the autologous tissue, can be different from the energy used to treat the area in need of regeneration where the autologous biologic is transplanted.

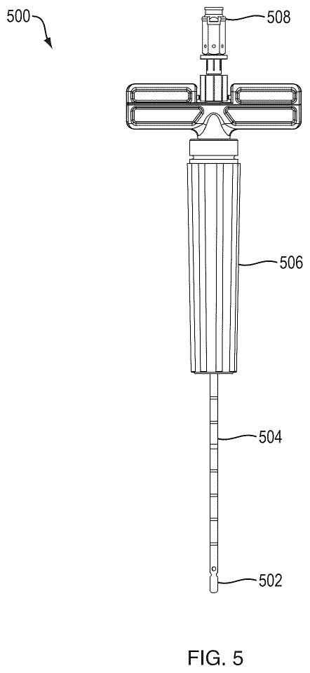

FIG. 6 depicts an example of an energy delivery device 600 that includes a side firing fiber. Multiple slots 602 and fibers 601 are shown. Each fiber aligns with a separate slot. This allows for multiple spots of target tissue to be treated simultaneously. Alternatively, a single fiber is used, and a series of micro prisms is applied in sequence to divert the optical energy from the fiber to the corresponding slots. The fiber that the energy is delivered over can be a side firing fiber where the fiber has an end cap with side ports that directs the energy more laterally and from the side. Alternatively, the cannula itself can be closed on the end with side ports. Insertion of the fiber into the cannula, after the cannula has been inserted into the body, forces the energy laterally to the orientation direction of the fiber. This feature allows the energy from the fiber to reach the area of the cartilage defect based on the anatomy of the joint space being treated (i.e. knee joint, hip joint, ankle joint, shoulder). This feature will also allow a more targeted delivery of the energy from the fiber to the damage created by sourcing the autologous biologic. For example, typically a cannula causes damage during the sourcing of the biologic. This wound heals from the outer edges of the hollow column shaped wound inward. The side firing laser allows the outer edges of the column shaped wound to be the target of the energy delivered. Multiple joints can be treated during the same procedure.

It is contemplated that other sources of energy can be used to prepare the damaged tissue such as ultrasound. The combination of directed pulsed energy within certain parameters, combined with a biologic graft is conducive to achieving optimal results.

Also, chronic disease in joints, specifically OA, involves the bone marrow on either side of the joint. In the case of knee OA, the bone on either side of the joint, i.e., tibia or femur is diseased with the cellular content of the marrow significantly degraded. This degraded marrow is partially responsible for a lack of nourishment in the joint. Treating this damaged marrow on the bone side of the joint will improve outcomes.

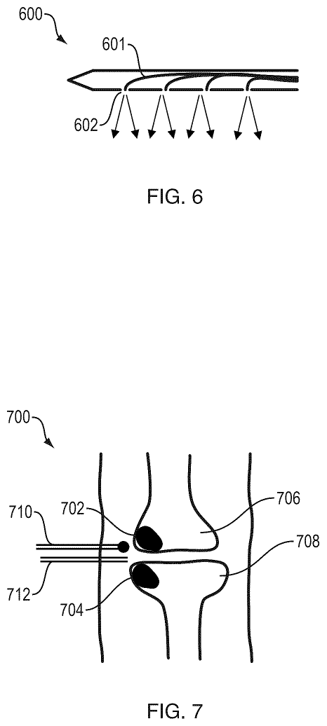

FIG. 7 schematically illustrates bone marrow edema 702 and 704 in a femur 706 and tibia 708, respectively, of a human knee joint 700. Bone edema can cause pain associated with cartilage disease. It is important to treat both sides of the joint, the bone side and the joint side. In the clinical examples, marrow was injected into the bone edema to address this malady. A device 710, e.g., an optical fiber, for delivering energy and a cannula 712 for delivering a biologic, e.g., marrow, are shown inserted into the joint 700. The energy delivery device 710 may be inserted separately from or in combination with the cannula 712.

Combining the sourcing of the autologous biologic such as adipose aspirate or bone marrow aspirate and the application of the energy delivered by the laser in novel ways, can improve the therapy. Allogeneic biologics are also contemplated such as placental tissue.

The biologic can be used to treat both the medullary side and intra articular side of a joint. The biologic is preferably autologous and can be marrow, blood, fat, or combinations or fractions thereof. For example, marrow, blood and fat can be centrifuged to select a fraction of the starting substrate to be used as the biologic treating composition. The biologic can serve as the graft or a separate graft material can be used. The separate graft material can be made of collagen or other biocompatible material. The graft material can be a product comprising a three-dimensional membrane and bound cultured chondrocytes. The graft material can be hydrated with a biologic such as marrow. The fiber can be delivered over a cannula and can be used to treat both the medullary and intra-articular side of the joint. The graft and or the biologic is exogeneous to the damaged tissue in need of repair. The laser can make nano fractional channels into the tissue such as cartilage, and the cells in the graft release proteins and growth factors that travel along the channels to nourish resident cells.

As noted above, a cannula can be used to introduce a fiber into the body. The fiber can be connected to a laser, the fiber being introduced to the body over the cannula. The energy delivered over the fiber can be in a range from 0.2 Hz to 1.2 Hz with a wavelength between 1.3 microns and 2.11 microns and a beam diameter between 300 and 900 microns and wattage between 1 and 5 Watts. Features incorporated into the tip of the fiber or the cannula allow the energy of the fiber to be delivered lateral to the trajectory of the cannula when it is first placed into the body and where the spot where the energy is being directed is determined through the use of guidance such as ultrasound. Multiple slots can be incorporated into the side firing tip or cannula to allow for simultaneous treatment of multiple areas along the cap or the cannula's length. A spacer can be incorporated into the cannula and or fiber so that the distance past the end of the cannula the fiber can travel is a fixed distance. Such spacer can be incorporated into the cannula and or fiber so that the orientation of the side port or ports can be determined after the tip of the cannula has been placed inside the body. In procedures where the fiber is delivered over a larger cannula or in an open procedure, a micro-array can be added to the tip of the fiber to allow multiple spots to be treated simultaneously in the same orientation as the direction of the fiber.

For example, a method to treat tissue of a patient can include using a laser where the energy is delivered over a fiber, such fiber being placed inside the body through a cannula. The cannula can be made of a shape memory alloy such as nitinol and can be configures to receive a stylet. When the stylet is positioned inside the cannula, the cannula is substantially straight but after placement of the cannula into the body and removal of the stylet, the cannula takes a pre-set bend. The cannula can be closed tipped with side ports. Multiple side ports spaced approximately 2 mm apart can be combined with a bundled fiber to treat multiple spots simultaneously. Multiple curved instruments are contemplated to deliver the fiber into various hard to reach zones in need of repair.

Embodiments can include features to regulate energy delivery, including features to prevent tissue damage due to overheating. Such features can include time intervals or other sensory mechanisms that limit the amount of heat the laser can generate. A fiber connected to a laser, the fiber being introduced to the body over a cannula, the energy delivered over the fiber can be regulated so that the time between treating cycles determined by the laser pulsing is set in a manner that even if the user were to make an error and treat the same spot twice, the tissue would not be heated over 70° C. A feedback mechanism can be incorporated into the fiber tip such that energy will not be delivered over the fiber unless the tip is approximately in contact with tissue and not fluid or air. Alternatively, thermal measurements can be used to access conditions of the tissue around the distal end of the fiber. Some embodiments may use thermal IR radiation of the heated tissue volume, while others may involve placing a direct temperature sensor at or near the distal end of the fiber.

A different source of energy is contemplated. For example, ultrasound can be delivered over a cannula, fiber, or stylet that is introduced into the body and positioned proximal to tissue in need of repair. A cannula can be used to introduce an ultrasound probe into the body or the probe can be delivered directly into the tissue. After the tissue is prepared by cavitation caused by the thermal mechanical energy delivered by ultrasound over the probe, a biologic can be used to supplement the treatment. Approximately the same energy delivered by the optical fiber is contemplated to be delivered by ultrasound to have an approximately similar impact on cartilage tissue. The energy delivered by ultrasound can also be pulsed in a similar manner or pattern as what was previously described for energy delivery by using the laser.

### EXEMPLIFICATION

### Example 1: Cartilage Graft Procedure

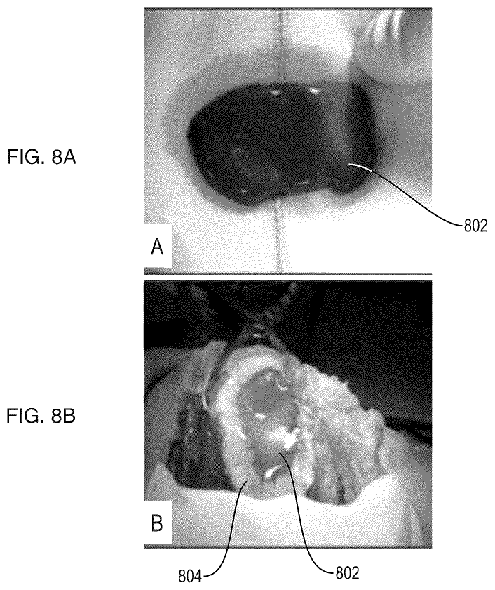

FIGS. 8A-8B depict a typical graft procedure in cartilage repair. In FIG. 8A, a graft 802 is shown being harvested from a donor site. In FIG. 8B, the graft 802 is shown placed at the target site 804. The graft can be hydrated with marrow or another biologic. The graft is irradiated prior to placement. The energy used to irradiate the graft is less than what is required to coagulate tissue. If the graft is hydrated at point of care with cells such as those contained in bone marrow aspirate, the graft can be irradiated prior to or just after hydration. To improve upon existing therapy, a device is disclosed that allows energy to be delivered interstitially where the fiber is in close proximity to the damaged tissue.

### Example 2: In Vitro Cartilage Illumination

FIG. 9A shows an example of temperature distribution and FIG. 9B shows an example of temperature dynamics when a laser illuminates cartilage in vitro. Parameters of the laser illumination are summarized in Table 1.

TABLE 1
Parameters of laser illumination using pulse sequence

| | |
|---|---|
| Power, W | 1 |
| Wavelength, μm | 1.56 |
| Pulse duration, s | 0.1 |
| Pause, s | 0.9 |
| Number of pulses | 10 |
| Pause between series, s | 10 |
| Number of series | 3 |
| Fiber diameter, μm | 600 |

### Example 3: Clinical Example—Animal

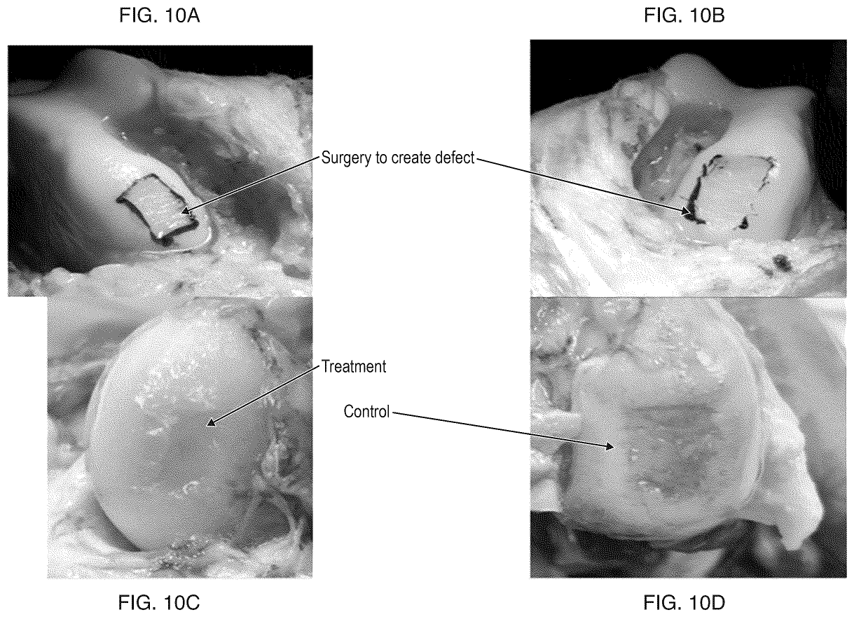

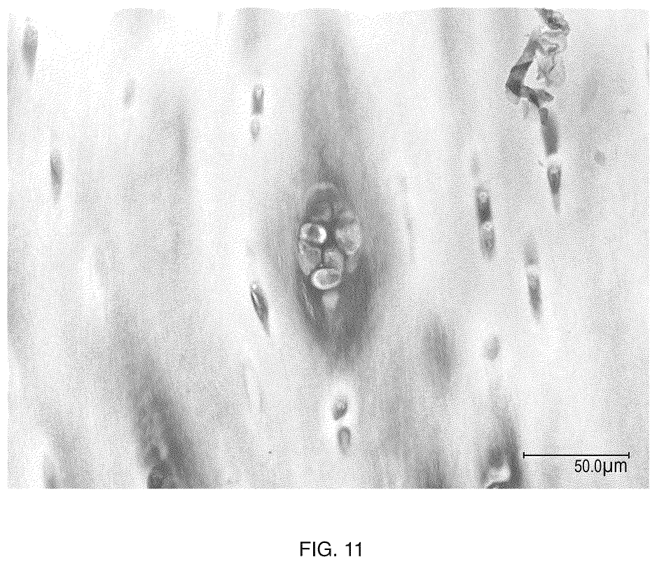

In a goat, two defects, each about 11 mm by 6 mm by 0.75 mm deep, were created on the cartilage of the medial femoral condyle (see FIGS. 10A-10B). After surgically creating the defects, the animal was allowed to move on the joints for a period of 60 days. After 60 days, one knee was accessed surgically and the other was left as a control. Marrow was aspirated from the sternum of the animal. In the surgically accessed knee, the defect created 60 days earlier was clearly visible. The defect was hydrated with marrow and irradiated using a laser and a fiber. The energy delivered was pulsed when the laser was activated. Each spot was treated by the laser for 50 seconds. During that 50 seconds, the laser was turned on for three intervals of 10 seconds and was turned off for two intervals of 10 seconds. The entire 50 seconds being a full sequence that treated a single spot. Nine spots were treated. After, irradiation, the animal was surgically closed and the joint capsule was filled with marrow aspirate (approximately 8 mL). After another 90 days the animal was sacrificed and histology was performed (see FIGS. 10C-10D). The histology showed hyaline like cartilage had formed over the defect on the treatment side (FIG. 10C) and not on the control side (FIG. 10D). Atomic microscopy showed the treatment side had many progenitor chondrocytes under the damaged tissue. Progenitor chondrocytes are typically a rare cell but they are responsible for maintaining and repairing cartilage. FIG. 11 shows a microscopic view of young cells in the treatment area.

Hyaline cartilage has type 2 collagen as its substrate; conversely, fibrotic tissue or scar tissue has type 1 collagen as its substrate. A surprising discovery from the work described herein is that fibrotic cartilage formation or scar tissue formation is caused primarily from tissue damage caused by the invasiveness of the procedure. For example, in the case of micro-fracture, the trauma caused by the drill penetrating through the cartilage, through the cortical plate and into the marrow space causes inflammation, bleeding and tissue damage. This trauma then leads to the formation of fibrotic cartilage. Conversely, use of a high energy laser to ablate and coagulate tissue also leads to this response by the body, inflammation and tissue damage. The hallmark of this unwanted tissue formation in the presence of inflammation is that the substrate is comprised of type 1 collagen or scar tissue/fibrotic tissue and not type 2 collagen or hyaline cartilage.

For cells to regenerate joint tissue (e.g., cartilage, ligaments or tendons) they need to be able to penetrate the tissue. The surface of cartilage is permeated by pores that are from 10 nanometers to 200 nanometers wide and deep. Cells and proteins can typically move into and out of those pores. When these pores become occluded, cells and proteins can no longer efficiently move into and out of the tissue to keep it healthy. This occlusion of pores then leads to osteoarthritis, for example. By creating pores on the scale of nanometers, 1 nanometer to 10,000 nanometers large with a preferable range of 30 to 100 nanometers, the ability to have cells and proteins permeate the tissue is restored. Specific pulsed energy delivered across the damaged tissue starting on the edges of healthy tissue appears to be needed for the therapy to be successful in creating micropores that will have a clinical benefit. The energy, pulsing, and distribution of the spots treated by the energy are described in greater detail elsewhere in this application. The creation of micropores through directed energy that does not damage tissue (i.e. does not create coagulation of tissue), results in a micro-environment where cells can penetrate the pores without the inflammatory signals created by tissue damage. Without these inflammatory signals, but with new pores available for tissue penetration, the cells and proteins make type 2 collagen and hyaline cartilage or other kinds of healthy tissue, but not scar tissue or type 1 collagen.

In addition, by targeting the directed pulsed energy against tissue where the acellular matrix tissue has a different density than the living cells embedded in the matrix tissue (the matrix is a dense stiff scaffold and the cells embedded in the scaffold are much less dense and malleable), the channels created are directed toward the less dense cells through the denser matrix. In histology, and after sacrificing the subject animal, it was observed through electron microscopy that there was a significant number of young progenitor chondrocytes in the pores created by the directed energy 90 days post creation of the micropores and transplantation of bone marrow. The number and types of cells were more than that were observed when the micropores alone are created without marrow transplantation. This lead to the observation that the progenitor chondrocytes came from cells transplanted with the marrow. Marrow cells became chondrocytes in this low inflammatory environment after creation of micropores. This observation is not typical when tissue damage and resultant inflammatory signals are combined with autologous marrow. The inflammation typically leads to fibrotic tissue formation.

A key insight is that altering the matrix tissue of the joint tissue with directed energy and without causing tissue damage and a corresponding local inflammatory response results in a healing cascade that does not produce scar tissue, and that combining delivery of a biologic that contains cells or proteins with this way to treat joint tissue significantly improves outcomes because the components of the biologic (cells and proteins) can contribute to scar-free healing in the absence of inflammatory signals. Inflammation free tissue regeneration leads to the formation of healthy new joint tissue with the absence of scar tissue, this process being greatly amplified when cells and proteins are transplanted as part of the therapy. Autologous biologics are preferred (e.g., PRP, marrow, fat) as they contain an abundance of cells and proteins.

### Example 4: Clinical Example—Human

A patient presented who 8 years previously had meniscal surgery to remove a bucket tear in conjunction with a full thickness ACL reconstruction. The patient's MRI results showed a thin weak ACL in the center of the repair, bone edema and damaged cartilage in the general area of the previous surgery. The patient's complaint was chronic pain that prevented daily activity. The patient, a male and in his early fifties, previously had his other knee replaced. He did not want a second knee replacement surgery if possible. This is typical of surgeries that remove meniscal tissue and arthroscopically repair joints through the removal of tissue. Approximately 1 million of these procedures are performed annually in the United States on knees. The common complaint is they predominantly lead to knee replacement surgery. This is equally as true when the procedure is performed on other joints. Insurance carriers may pressure clinicians to skip this procedure and go straight to joint replacement but patients want to avoid total joint replacement for as long as possible. However, for certain joints, replacement is not an option such as ankles and elbows. For this patient, the clinician aspirated marrow, used ultrasound to see inside the joint, placed the tip of the fiber against the damaged area of the joint, irradiated several spots using pulsed and timed delivery of energy similar to the animal model, delivered bone marrow to the edema in the marrow under the surface of the defect of the cartilage (sub-chondral), and filled the joint capsule with marrow after the procedure. After 6 months, the patient reported a dramatic reduction of pain and full return to normal activity. Comparing MRI results pre- and post-procedure demonstrated a dramatic improvement in bone edema and joint thickening in the area of the greatest cartilage deficiency.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A device for treatment of tissue in a joint of a mammal, comprising:
an energy source;
an energy-delivery implement coupled to the energy source and having a distal portion configured to be inserted into the joint, the distal portion having at least one energy-emitting portion; and
a control module causing the energy source to produce energy in a pre-defined treatment sequence, the sequence comprising at least two pulses of energy separated by an interval, the energy-delivery implement emitting the energy at the distal portion, to create a zone of thermal stress within target tissue, without causing substantial coagulation of the target tissue, wherein the size of the thermal stress zone is substantially less than the size of the target tissue being treated.

2. The device of claim 1, wherein the energy is optical energy.

3. The device of claim 2, wherein the energy-delivery implement includes an optical fiber.

4. The device of claim 3, wherein the energy-delivery implement is configured to create plural thermal stress zones simultaneously.

5. The device of claim 4, wherein the energy-delivery implement is a bundle of optical fibers.

6. The device of claim 4, wherein the energy-delivery implement is a multi-core optical fiber.

7. The device of claim 4, wherein a micro-array is attached to an end of the fiber allowing for plural thermal stress zones to be created simultaneously.

8. The device of claim 1, wherein the energy source is a laser.

9. A method to treat damaged tissue in a joint of a mammal, comprising:
providing a treatment device including an energy source and an energy-delivery implement;
inserting the energy-delivery implement into the joint;
positioning an energy-emitting portion of the implement in the proximity of a first target spot in a target zone of the damaged tissue;
activating the energy source of treatment device to deliver a treatment sequence of energy at the first target spot, the sequence comprising at least two pulses of energy separated by an interval, to create a zone of thermal stress within target tissue, without causing substantial coagulation of the tissue;
relocating the energy-delivery implement to position the energy-emitting portion in the proximity of another target spot of the target zone, located from the first target spot at a distance not shorter than a radius of the zone of thermal stress; and
repeating the activating and relocating until energy is delivered to all target spots of the target zone.

10. The method of claim 9, wherein the distance between two neighboring target spots exceeds 1 mm.

11. The method of claim 9, wherein at least two target spots are treated simultaneously.

12. The method of claim 11, wherein the energy-delivery implement includes a bundle of fibers or a micro-array.

13. The method of claim 9, wherein the duration of time the energy is delivered at a single target spot is in a range of about 10 seconds to about 5 minutes.

14. The method of claim 13, wherein the duration is about 50 seconds.

15. The method of claim 13, wherein the energy delivered can be turned on and off while the energy-delivery implement is held at a single location with a time interval between when the energy is turned off and when it is turned on being in a range of about 1 second to about 1 minute.

16. The method of claim 15, wherein the time interval is about 10 seconds.

17. The method of claim 9, wherein the energy-delivery implement includes a micro-array and wherein the energy emitted is under 3 watts of power per target spot treated by the micro-array and the size is 900 microns or less for each target spot.

18. The method of claim 9, wherein the pulse repetition frequency is in a range of about 0.2 Hz to about 3.2 Hz.

19. The method of claim 9, wherein the energy source is a laser and the wavelength of the energy is in a range of about 0.6 microns to about 2.1 microns.

20. The method of claim 19, wherein the wavelength is in a range of 1.3 microns to 1.65 microns.

21. The method of claim 9, wherein the energy-delivery implement includes a micro-array head that contains plural lenses with centers positioned greater than 0.05 millimeters apart and less than 5 millimeters apart.

22. The method of claim 21, wherein the lens centers are about 0.5 millimeters apart.

23. The method of claim 9, wherein the energy-delivery implement includes an array of plural diffractive elements, centers of the elements positioned greater than 0.05 millimeters apart and less than 5 millimeters apart.

24. The method of claim 23, wherein the centers of the diffractive elements are about 0.5 millimeters apart.

25. The method of claim 9, further comprising:
identifying a location of the damaged tissue in the joint using a diagnostic device; and
identifying the target zone to be treated at the location of the damaged tissue, the target zone containing plural target spots.

26.-43. (canceled)
